(19) United States
(12) Patent Application Publication
(10) Pub. No.: US 2023/0357699 A1
(43) Pub. Date: Nov. 9, 2023

(54) Cell Culture System

(71) Applicants: Terumo Kabushiki Kaisha, Tokyo (JP); Terumo BCT, Inc., Lakewood, CO (US)

(72) Inventors: Masatsugu IGARASHI, Kanagawa (JP); Hirotaka OHASHI, Tokyo (JP); Thomas J. FELT, Boulder, CO (US); Dennis J. HLAVINKA, Arvada, CO (US)

(21) Appl. No.: 18/215,947

(22) Filed: Jun. 29, 2023

Related U.S. Application Data
(63) Continuation of application No. PCT/JP2022/012950, filed on Mar. 22, 2022.

(30) Foreign Application Priority Data
Mar. 26, 2021 (JP) 2021-053065

(51) Int. Cl. C12M 1/34; C12M 1/00; C12M 3/00; C12M 1/36

(52) U.S. Cl. CPC C12M 41/12; C12M 29/16; C12M 23/48; C12M 41/48

(57) ABSTRACT

A cell culturing system includes a processing unit that cultures cells, a reactor installation device in which the processing unit is installable, a connection circuit connected to the processing unit, a circuit control device to and from which the connection circuit is attachable and detachable, and a tank device including a culture medium accommodation unit for accommodating the culture medium. The processing unit includes a plurality of bioreactors. The culture medium in the culture medium accommodation unit is supplied to the processing unit via the connection circuit. The culture medium accommodation unit is capable of accommodating the culture medium of an amount required for culturing the cells in the processing unit.

### CROSS-REFERENCE TO RELATED APPLICATIONS

The present application is a continuation application of the International Patent Application No. PCT/JP2022/012950 filed on Mar. 22, 2022, which designated the U.S. and claims the benefit of priority from Japanese Patent Application No. JP2021-053065 filed on Mar. 26, 2021. The entire disclosures of the above-identified applications are incorporated herein by reference.

### FIELD

The present disclosure relates to a cell culturing system.

### BACKGROUND

A cell culturing device may include a reactor installation unit that is configured to receive one bioreactor that cultures cells. The cell culturing device may also include a circuit control unit to and from which a connection circuit connected to the bioreactor may be attachable and detachable. The circuit control unit may be configured to supply cells and a culture medium from the connection circuit to the bioreactor and to collect cultured cells from the bioreactor.

The reactor installation unit and the circuit control unit may be integrally formed. In such instances and where it is desired to increase an amount of cell culture, it may necessary to prepare a plurality of cell culturing devices including circuit control units for each bioreactor, which can increase costs.

Accordingly, there is a need for a cell culturing system that is capable of efficiently increasing the amount of cell culture while suppressing cost increases.

### SUMMARY

In at least one example embodiment, the present disclosure provides a cell culturing system that includes a processing unit that cultures cells, a reactor installation device in which the processing unit is installable, a connection circuit connected to the processing unit, and a circuit control device to and from which the connection circuit is attachable and detachable. The circuit control device may be configured to supply the cells and a culture medium from the connection circuit to the processing unit. The circuit control device may also be configured to collect cultured cells from the processing unit and moving the cultured cells to the connection circuit. The cell culturing system may also include a tank device. The tank device may include a culture medium accommodation unit that is configured to accommodate the culture medium. The processing unit may include a plurality of bioreactors. The culture medium in the culture medium accommodation unit may be supplied to the processing unit via the connection circuit. The culture medium accommodation unit may be capable of accommodating the culture medium of an amount required for culturing the cells in the processing unit.

In at least one example embodiment, it may be sufficient to prepare the circuit control device for each processing unit, so that an overall number of the circuit control devices is less than the number of the bioreactors. Accordingly, it is possible to efficiently increase the amount of cell culture while suppressing an increase in cost. Because the culture medium accommodation unit may accommodate the culture medium of the amount required for culturing the cells in the processing unit, even when a large amount of culture medium is required at the time of cell culture using the plurality of bioreactors, it may not be required to replace the culture medium accommodation unit during the cell culture.

### DESCRIPTION OF DRAWINGS

FIG. 1 is a schematic illustrating an example cell culturing system in accordance with at least one example embodiment of the present disclosure.

FIG. 2 is a circuit configuration diagram of the example cell culturing system as illustrated in FIG. 1 in accordance with at least one example embodiment of the present disclosure.

FIG. 3 is a circuit configuration diagram of the processing unit and its surroundings as introduced in FIG. 2 in accordance with at least one example embodiment of the present disclosure.

FIG. 4 is a cross-sectional view of the tank device in as introduced in FIG. 1 in accordance with at least one example embodiment of the present disclosure.

FIG. 5 is a cross-sectional view taken along line V-V of FIG. 4.

FIG. 6 is a perspective view of the circuit control device and the reactor installation device as introduced in FIG. 1 in accordance with at least one example embodiment of the present disclosure.

FIG. 7 is a perspective view of the circuit control device in accordance with at least one example embodiment of the present disclosure.

FIG. 8 is a flowchart of a cell culturing method that uses, for example, the example cell culturing system illustrated in FIG. 1, in accordance with at least one example embodiment of the present disclosure.

FIG. 9 is a schematic another example cell culturing system that includes a cell culturing device in accordance with at least one example embodiment of the present disclosure.

FIG. 10 is a schematic another example cell culturing system in accordance with at least one example embodiment of the present disclosure.

FIG. 11 is a circuit configuration diagram for the example cell culturing system illustrated in FIG. 10 in accordance with at least one example embodiment of the present disclosure.

FIG. 12 is a partial cross-sectional explanatory view of the tank device as introduced in FIG. 10 in accordance with at least one example embodiment of the present disclosure.

### DETAILED DESCRIPTION

Example embodiments of the present disclosure are hereafter described with reference to the accompanying drawings.

A cell culturing system 10 in accordance with at least one example embodiment of the present disclosure may be configured to culture (e.g., propagate) cells that have been separated from biological tissue.

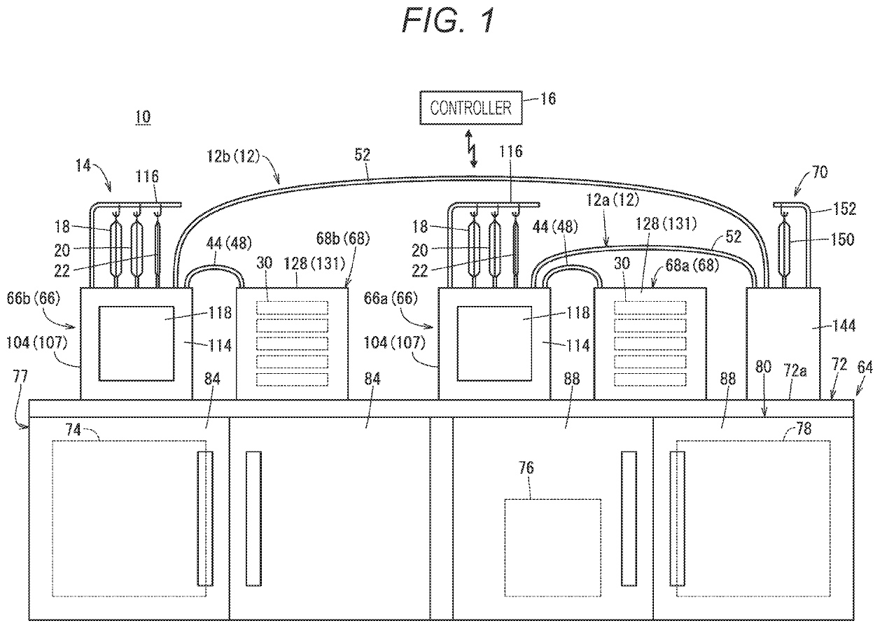

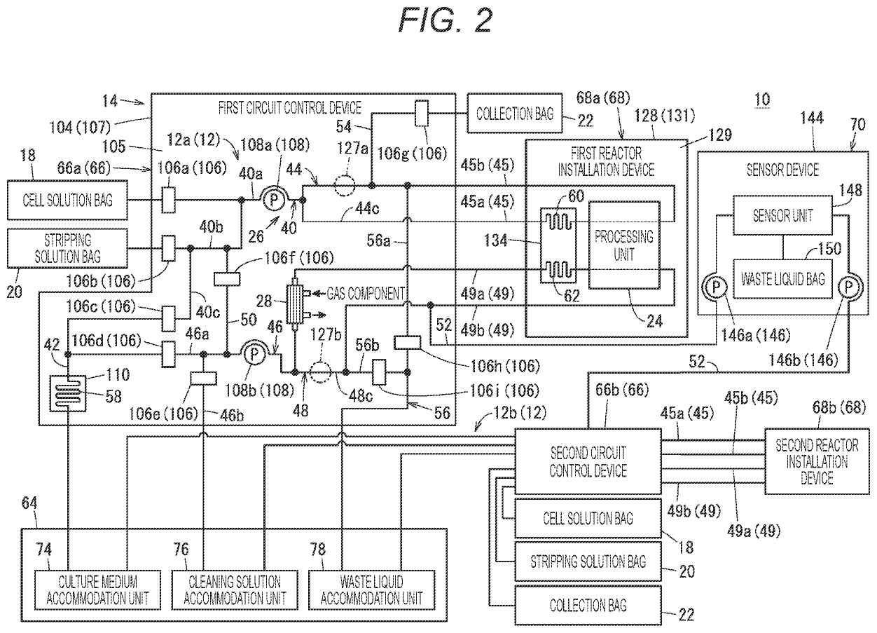

As illustrated in FIGS. 1 and 2, the cell culturing system 10 may include two cell culturing kits 12 though which a liquid may flow, a cell culturing device 14 in which the two cell culturing kits 12 are set, and a controller 16. The two cell culturing kits 12 may include a first cell culturing kit 12a and a second cell culturing kit 12b. The first cell culturing kit 12a and the second cell culturing kit 12b may be the. In the following description, when the first cell culturing kit 12a and the second cell culturing kit 12b are not especially distinguished from each other, they may be simply referred to as the cell culturing kit 12.

The liquid that flows in the cell culturing kit 12 may include, for example, a solution that includes cells (i.e., a cell solution), a culture medium for propagating the cells (i.e., a culture solution), a cleaning solution that cleans the interior of the cell culturing kit 12, a stripping solution for stripping the cells, and/or the like.

The cells for culture may include cells contained in the blood (such as, T cells and/or the like) and/or stem cells (such as, ES cells, iPS cells, mesenchymal stem cells, and/or the like). The culture medium may be selected according to the cells of a living body. In at least one example embodiment, the culture medium may be prepared by adding various amino acids, vitamins, serum, and the like to a basic solution. The basic solution may include a balanced salt solution (BSS). The cleaning solution may include a buffer solution and/or a physiological saline. The buffer solution may include, for example, phosphate buffered salts (PBS), tris-buffered saline (TBS), and/or the like. The stripping solution may include, for example, trypsin, ethylenediaminetetraacetic acid (EDTA) solution, and/or the like.

As illustrated in FIG. 2, the cell culturing kit 12 may include a cell solution bag 18, a stripping solution bag 20, a collection bag 22, a processing unit 24, a connection circuit 26, and/or a gas exchanger 28.

The cell solution bag 18, the stripping solution bag 20, and/or the collection bag 22 may include a flexible material that is shaped to form a bag. The flexible material may include a soft resin, such as polyvinyl chloride and/or polyolefin.

The cell solution bag 18 may be configured to accommodate the cell solution. The stripping solution bag 20 may be configured to accommodate the stripping solution. The collection bag 22 may be configured to accommodate the cultured cells. Before the cell culturing kit 12 is used, the collection bag 22 may be an empty bag in which no liquid is accommodated.

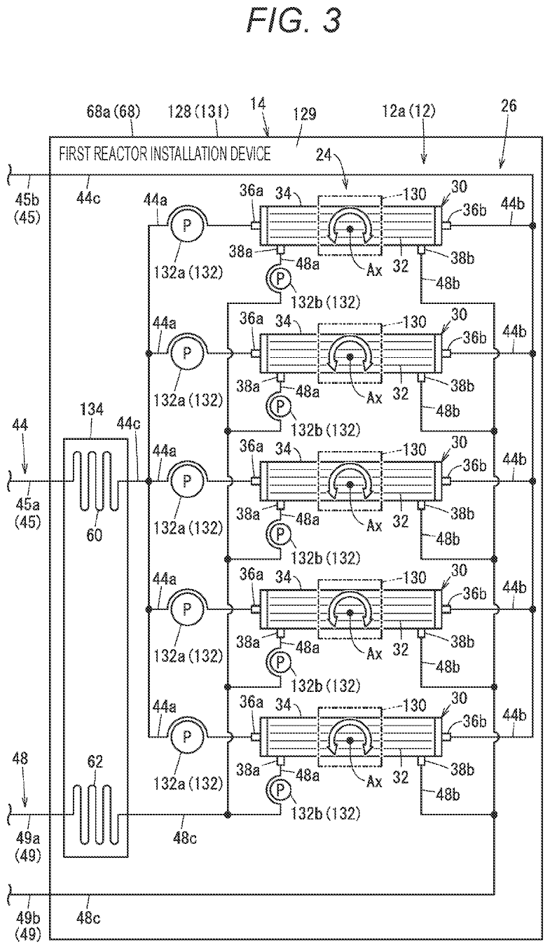

As illustrated in FIG. 3, the processing unit 24 may include five bioreactors 30 arranged in parallel. In at least one example embodiment, the five bioreactors 30 may have the same configuration. In other example embodiment, the five bioreactors 30 may be different from one another in size, shape, and/or the like. The bioreactor 30 may be formed as a hollow fiber bioreactor. The bioreactor 30 may include a large number (plurality) of hollow fibers 32 and a cylindrical housing 34 that accommodates the hollow fibers 32.

The hollow fiber 32 may extended in a longitudinal direction of the housing 34. Both ends of the hollow fiber 32 may be opened. One end of the hollow fiber 32 may be fixed to one end of the housing 34. The other end of the hollow fiber 32 may be fixed to the other end of the housing 34. A plurality of pores not illustrated may be formed on a wall part that forms the hollow fiber 32. An inner cavity of the hollow fiber 32 may be referred to as an intra capillary (IC) region. The intra capillary (IC) region may communicate with an extra capillary (EC) region located outside the hollow fiber 32 in the housing 34 via the pores. The pores may be sizes to allow small molecules (including, for example, water, ions, oxygen, lactate, and/or the like) to pass therethrough while preventing the passage of larger molecules or macromolecules (including, for example, cell and/or the like) therethrough. In at least one example embodiment, the diameter of the pores may range from about 0.005 micrometers (μm) to about 10 μm.

The hollow fiber 32 may be formed from polyolefin resins and/or polymer materials. The polyolefin resins may include, for example, polypropylene and/or polyethylene. The polymer materials may include, for example, polysulfone, polyethersulfone, polyacrylonitrile, polytetrafluoroethylene, polystyrene, polymethylmethacrylate, cellulose acetate, cellulose triacetate, and/or regenerated cellulose.

The housing 34 may include an IC inlet port 36a, an IC outlet port 36b, an EC inlet port 38a, and/or an EC outlet port 38b. The IC inlet port 36a may be provided at one end of the housing 34. The IC inlet port 36a may be configured to introduce the liquid (e.g., the cell solution, culture medium, cleaning solution, and/or stripping solution) guided from the connection circuit 26 (an IC circulation circuit 44) to and into the IC region of the bioreactor 30. The IC outlet port 36b may be provided at the other end of the housing 34. The IC outlet port 36b may be configured to lead the liquid from the IC region of the bioreactor 30 to the connection circuit 26 (IC circulation circuit 44).

The EC inlet port 38a and the EC outlet port 38b may be provided on an outer peripheral surface of the housing 34. The EC inlet port 38a may be configured to introduce the liquid (e.g., the culture medium and/or the cleaning solution) as guided from the connection circuit 26 (an EC circulation circuit 48) to and into the EC region of the bioreactor 30. The EC outlet port 38b may be configured to lead the liquid from the EC region of the bioreactor 30 to the connection circuit 26 (the EC circulation circuit 48).

As illustrated, for example, in FIG. 2, the connection circuit 26 may be extended linearly. The connection circuit 26 may include a soft resin material that is formed, for example, into a tubular shape. In at least one example embodiment, the connection circuit 26 may be formed, for example, by stacking two sheets on each other in a thickness direction and joining (e.g., fusing, sealing) sites other than a portion that serves as a flow path. A wall part (non-sealed portion) that forms the connection circuit 26 may be formed to protrude outward from the sealed portion in such a manner that the connection circuit 26 becomes an opened flow path in a natural state. Extra sheets on sides of the flow path of the connection circuit 26 may be cut off. The connection circuit 26 may include an IC supply flow path 40, a culture medium supply line 42, the IC circulation circuit 44, an EC supply flow path 46, the EC circulation circuit 48, a coupling line 50, a sampling line 52, a collection line 54, and/or a waste liquid flow path 56.

The IC supply flow path 40 may include a first IC supply line 40a, a second IC supply line 40b, and/or a third IC supply line 40c. One end of the first IC supply line 40a may be aseptically joined to the cell solution bag 18. The other end of the first IC supply line 40a may be coupled to the IC circulation circuit 44. One end of the second IC supply line 40b may be aseptically joined to the stripping solution bag 20. The other end of the second IC supply line 40b may be coupled to an intermediate portion of the first IC supply line 40a. One end of the third IC supply line 40c may be coupled to the culture medium supply line 42. The other end of the third IC supply line 40c may be coupled to an intermediate portion of the second IC supply line 40b.

One end of the culture medium supply line 42 may be aseptically joined to a connection tube of a culture medium accommodation unit 74 of the cell culturing device 14 when the cell culturing kit 12 is set in the cell culturing device 14. The other end of the culture medium supply line 42 may be coupled to the third IC supply line 40c. The culture medium supply line 42 may be provided with a culture medium intermediate flow path 58 for raising temperature of the culture medium (cooled culture medium) guided from the culture medium accommodation unit 74 to desired temperature. The culture medium intermediate flow path 58 may be provided between the culture medium accommodation unit 74 and the processing unit 24.

As illustrated, for example, in FIGS. 2 and 3, the IC circulation circuit 44 may be configured to circulate the liquid introduced from the IC supply flow path 40 to and into the IC circulation circuit 44 in the IC region of each bioreactor 30. As illustrated, for example, in FIG. 3, the IC circulation circuit 44 may include five IC introduction lines 44a, five IC lead-out lines 44b, and an IC circulation line 44c.

The five IC introduction lines 44a may be coupled to the IC inlet ports 36a of the five bioreactors 30. The five IC lead-out lines 44b may be coupled to the IC outlet ports 36b of the five bioreactors 30. One end of the IC circulation line 44c may be coupled to the five IC introduction lines 44a. The other end of the IC circulation line 44c may be coupled to the five IC lead-out lines 44b. The IC circulation line 44c may be provided with an IC intermediate flow path 60 for raising temperature of the liquid that flows through the IC circulation line 44c to desired temperature.

As illustrated, for example, in FIG. 2, the EC supply flow path 46 may include a first EC supply line 46a and a second EC supply line 46b. One end of the first EC supply line 46a may be coupled to the culture medium supply line 42. The other end of the first EC supply line 46a may be coupled to the EC circulation circuit 48. One end of the second EC supply line 46b may be aseptically joined to a connection tube of a cleaning solution accommodation unit 76 of the cell culturing device 14 when the cell culturing kit 12 is set in the cell culturing device 14. The other end of the second EC supply line 46b may be coupled to an intermediate portion of the first EC supply line 46a.

As illustrated, for example, in FIGS. 2 and 3, the EC circulation circuit 48 may be configured to circulate the liquid guided from the EC supply flow path 46 to the EC circulation circuit 48 in the EC region of each bioreactor 30. In FIG. 3, the EC circulation circuit 48 may include five EC introduction lines 48a, five EC lead-out lines 48b, and an EC circulation line 48c.

The five EC introduction lines 48a may be coupled to the EC inlet ports 38a of the five bioreactors 30. The five EC lead-out lines 48b may be coupled to the EC outlet ports 38b of the five bioreactors 30. One end of the EC circulation line 48c may be coupled to the five EC introduction lines 48a. The other end of the EC circulation line 48c may be coupled to the five EC lead-out lines 48b. The EC circulation line 48c may be provided with an EC intermediate flow path 62 for raising temperature of the liquid that flows through the EC circulation line 48c to desired temperature.

As illustrated, for example, in FIG. 2, the coupling line 50 may be configured to couple the IC supply flow path 40 to the EC supply flow path 46. For example, one end of the coupling line 50 may be coupled to the second IC supply line 40b downstream from a coupling portion to the third IC supply line 40c. The other end of the coupling line 50 may be coupled to the first EC supply line 46a downstream from a coupling portion to the second EC supply line 46b on.

The sampling line 52 may include a flow path for obtaining a part of the culture medium that flows through the EC region of each bioreactor 30. One end of the sampling line 52 may be coupled to the EC circulation line 48c downstream side from the processing unit 24. The other end of the sampling line 52 may be aseptically joined to a connection tube of a sensor device 70 of the cell culturing device 14 when the cell culturing kit 12 is set in the cell culturing device 14. In at least one example embodiment, one end of the sampling line 52 may be provided on a circuit control device 66 in a set state (refer to FIG. 1). In at least one example embodiment, the one end of the sampling line 52 may also be provided on a reactor installation device 68 in the set state.

The collection line 54 may include a flow path for guiding the cultured cells from the IC circulation circuit 44 to the collection bag 22. One end of the collection line 54 may be coupled to the IC circulation line 44c downstream from the processing unit 24. The other end of the collection line 54 may be aseptically joined to the collection bag 22.

The waste liquid flow path 56 may include a flow path for guiding a used liquid (waste liquid) to a waste liquid accommodation unit 78 of the cell culturing device 14. The waste liquid flow path 56 may include an IC waste liquid line 56a and an EC waste liquid line 56b. One end of the IC waste liquid line 56a may be coupled to the IC circulation line 44c between the processing unit 24 and a coupling portion to the collection line 54. The other end of the IC waste liquid line 56a may be aseptically joined to a connection tube of the waste liquid accommodation unit 78 when the cell culturing kit 12 is set in the cell culturing system 10. One end of the EC waste liquid line 56b may be coupled to the EC circulation line 48c between a coupling portion to the sampling line 52 and a coupling portion to the first EC supply line 46a. The other end of the EC waste liquid line 56b may be coupled to the IC waste liquid line 56a.

The gas exchanger 28 may be provided on the EC circulation line 48c between a coupling portion to the first EC supply line 46a and the EC intermediate flow path 62. The gas exchanger 28 may be configured to mix a predetermined gas component with the liquid (e.g., culture medium) that flows through the EC circulation line 48c. The gas component may include, for example, components similar to natural air (e.g., nitrogen N₂: 75%, oxygen O₂: 20%, and/or carbon dioxide CO₂: 5%).

The structure of the gas exchanger 28 is not limited. For example, in at least one example embodiment, a structure in which a plurality of hollow fibers 32 is provided in the housing 34 may be used, as is the case with the bioreactor 30.

As illustrated, for example, in FIGS. 1 and 2, the cell culturing device 14 may include one tank device 64, two circuit control devices 66, two reactor installation devices 68, and/or one sensor device 70. The two circuit control devices 66 may include a first circuit control device 66a and a second circuit control device 66b. The two reactor installation devices 68 may include a first reactor installation device 68a and a second reactor installation device 68b. In the instance where the first circuit control device 66a and the second circuit control device 66b are not especially distinguished from each other, they may be simply referred to as the circuit control device 66. In the instance where the first reactor installation device 68a and the second reactor installation device 68b are not especially distinguished from each other, they may be simply referred to as the reactor installation device 68.

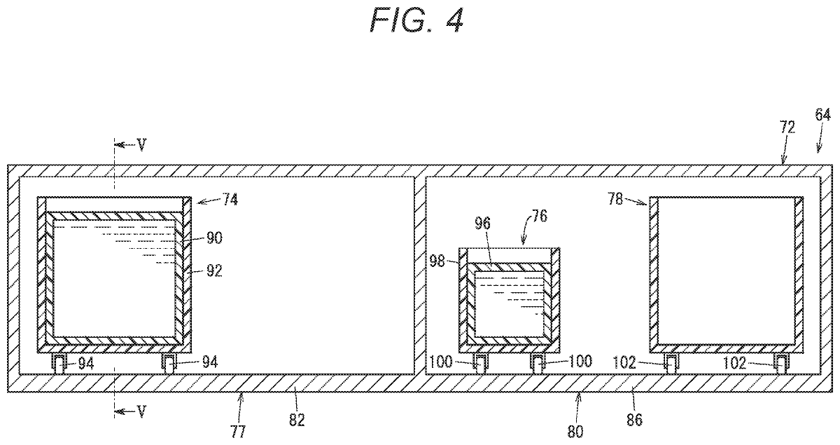

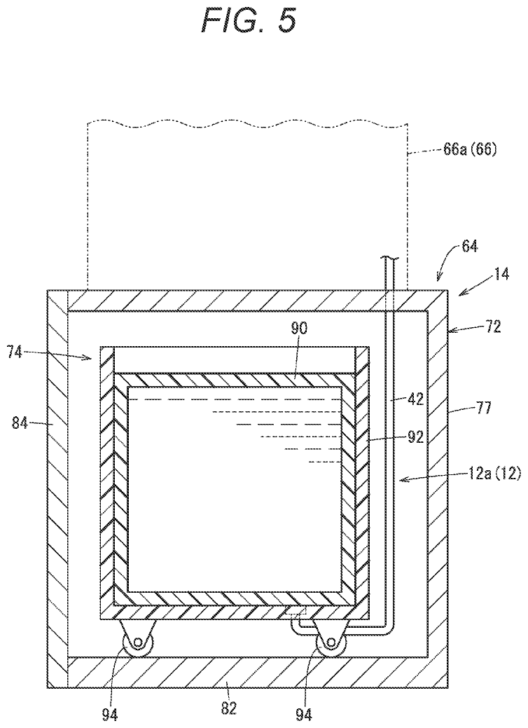

As illustrated, for example, in FIGS. 1 and 4, the tank device 64 may include a box-shaped base 72 installed, for example, on a floor surface. The tank device 64 may also include the culture medium accommodation unit 74 in which the culture medium is accommodated, the cleaning solution accommodation unit 76 in which the cleaning solution is accommodated, and the waste liquid accommodation unit 78 capable of accommodating the waste liquid. The base 72 may include a first case unit 77 and a second case unit 80. The first case unit 77 may include a first case body 82 in which the culture medium accommodation unit 74 may be arranged. A first door 84 (refer to FIGS. 1 and 5) may be provided on a front surface of the first case body 82 so as to be openable and closable.

The first case unit 77 may include a cooling unit that cools the culture medium to desired temperature (for example, ranging from about 4° C. to about 8° C.). The second case unit 80 may include a second case body 86 in which the cleaning solution accommodation unit 76 and the waste liquid accommodation unit 78 may be arranged. A second door 88 (refer to FIG. 1) may be provided on a front surface of the second case body 86 so as to be openable and closable. The second case unit 80 may not have a cooling function.

As illustrated, for example, in FIGS. 4 and 5, the culture medium accommodation unit 74 may include a culture medium tank 90 molded, for example, of a hard resin into a box shape and a culture medium installing member 92 capable of accommodating the culture medium tank 90. In at least one example embodiment, the culture medium tank 90 may be a disposable product. In other example embodiments, the culture medium tank 90 may be a reusable product. The culture medium supply line 42 of each cell culturing kit 12 may be connected to the culture medium tank 90 when the cell culturing kit 12 is set in the cell culturing device 14 (referred to as the “set state”). That is, the culture medium accommodation unit 74 (culture medium tank 90) may be shared by the two processing units 24 (two cell culturing kits 12) any may supply the culture medium from the culture medium accommodation unit 74 to the two processing units 24 via the two connection circuits 26.

The culture medium tank 90 may be configured to accommodate the culture medium in an amount required for culturing the cells in the two processing units 24 (two cell culturing kits 12). The culture medium tank 90 may be configured to accommodate the culture medium in the amount required for culturing the cells by the two cell culturing kits 12 (ten bioreactors 30) coupled to the culture medium tank 90. For example, in a case where about 20 L of culture medium is required for one bioreactor 30, about 200 L of culture medium may be accommodated in the culture medium tank 90. When the culture medium of the amount required from the start to the end of the cell culture is accommodated in advance in the culture medium tank 90, replacement of the culture medium accommodation unit 74 may not be required, improving efficiency. The culture medium may be added to in the culture medium tank 90 in a clean bench.

When the culture medium is stored at room temperature (for example, about 22° C.) or in a bright place continuously for a cell culturing period (for example, greater than or equal to about seven days), there may be a risk that components of the culture medium (proteins, glutamine, and/or the like) may be denatured. However, in at least one example embodiment of the present disclosure, the culture medium may be stored in the first case unit 77 at low temperature and in a dark place, such that the denaturation of the components of the culture medium may be suppressed.

The culture medium installing member 92 may be molded of a hard resin. The culture medium installing member 92 may be a reusable product that may be reused. An upper side of the culture medium installing member 92 may be opened. A plurality of rollers 94 (wheels) may be provided on a bottom surface of the culture medium installing member 92. As a result, the culture medium accommodation unit 74, which is relatively heavy, may be moved in a state in which the culture medium tank 90 is arranged inside the culture medium installing member 92. Therefore, it is possible to easily and efficiently take the culture medium accommodation unit 74 in and out of the first case unit 77. It should be appreciated, however, that in other embodiments, the culture medium installing member 92 may include a cart.

As illustrated, for example, in FIG. 4, the cleaning solution accommodation unit 76 may include a cleaning solution tank 96 molded, for example, of a hard resin into a box shape and a cleaning solution installing member 98 capable of accommodating the cleaning solution tank 96. In at least one example embodiment, the cleaning solution tank 96 may be a disposable product. In other example embodiments, the cleaning solution tank 96 may be a reusable product. The second EC supply line 46b of each cell culturing kit 12 may be connected to the cleaning solution tank 96 in the set state. That is, the cleaning solution accommodation unit 76 (cleaning solution tank 96) may be shared by the two processing units 24 (two cell culturing kits 12). The cleaning solution may be supplied from the cleaning solution accommodation unit 76 to the two processing units 24 via the two connection circuits 26.

The cleaning solution tank 96 may accommodate the culture medium in an amount required for cleaning the two processing units 24 (two cell culturing kits 12). The cleaning solution tank 96 may accommodate the cleaning solution of the amount required for cleaning the two cell culturing kits 12 coupled to the cleaning solution tank 96. As such, it is not required to replace the cleaning solution tank 96 during the cell culture, improving efficiency

The cleaning solution installing member 98 may be molded, for example, of a hard resin. The cleaning solution installing member 98 may be a reusable product. An upper side of the cleaning solution installing member 98 may be opened. A plurality of rollers 100 (wheels) may be provided on a bottom surface of the cleaning solution installing member 98. As a result, the cleaning solution accommodation unit 76, which is relatively heavy, may be moved in a state in which the cleaning solution tank 96 is arranged inside the cleaning solution installing member 98. Therefore, it may be possible to easily and efficiently take the cleaning solution accommodation unit 76 in and out of the second case unit 80. It should be appreciated, however, that in other embodiments, the cleaning solution installing member 98 may include a cart.

The waste liquid accommodation unit 78 may be molded, for example, of a hard resin into a box shape. In at least one example embodiment, the waste liquid accommodation unit 78 may be a reusable product. In other example embodiments, the waste liquid accommodation unit 78 may be a disposable product. The waste liquid flow path 56 (IC waste liquid line 56a) of each cell culturing kit 12 may be connected to the waste liquid accommodation unit 78 in the set state. That is, the waste liquid accommodation unit 78 may be shared by the two processing units 24 (two cell culturing kits 12) in order to discharge the waste liquid from the two processing units 24 to the waste liquid accommodation unit 78 via the two connection circuits 26.

The waste liquid accommodation unit 78 may accommodate the waste liquid discharged from the two processing units 24 (two cell culturing kits 12). That is, the waste liquid accommodation unit 78 may be formed to have a size capable of accommodating the waste liquid (liquid) used in the two cell culturing kits 12 as coupled to the waste liquid accommodation unit 78. In such instances, it may not be required to replace the waste liquid accommodation unit 78 during the cell culture, so that it is efficient.

A plurality of rollers 102 (wheels) may be provided on a bottom surface of the waste liquid accommodation unit 78. As a result, the waste liquid accommodation unit 78 may be easily moved by the plurality of rollers 102. Therefore, it is possible to easily and efficiently take the waste liquid accommodation unit 78 in and out of the second case unit 80.

The culture medium tank 90 and the cleaning solution tank 96 are not limited to the above examples formed of, for example, of the hard resin. In at least one example embodiment, the culture medium tank 90 and the cleaning solution tank 96 may include, for example, large-capacity bags formed, for example, of a soft resin into a bag shape.

As illustrated, for example, in FIG. 1, the first circuit control device 66a, the first reactor installation device 68a, the second circuit control device 66b, the second reactor installation device 68b, and/or the sensor device 70 may be arranged on an upper surface 72a of the base 72. The first circuit control device 66a and the first reactor installation device 68a may be adjacent to each other. The second circuit control device 66b and the second reactor installation device 68b may be adjacent to each other.

The connection circuit 26 of the first cell culturing kit 12a may be attachable to and detachable from the first circuit control device 66a. The first circuit control device 66a may be for supplying the cells and the culture medium from the connection circuit 26 to the processing unit 24 and collecting the cultured cells from the processing unit 24 to the connection circuit 26.

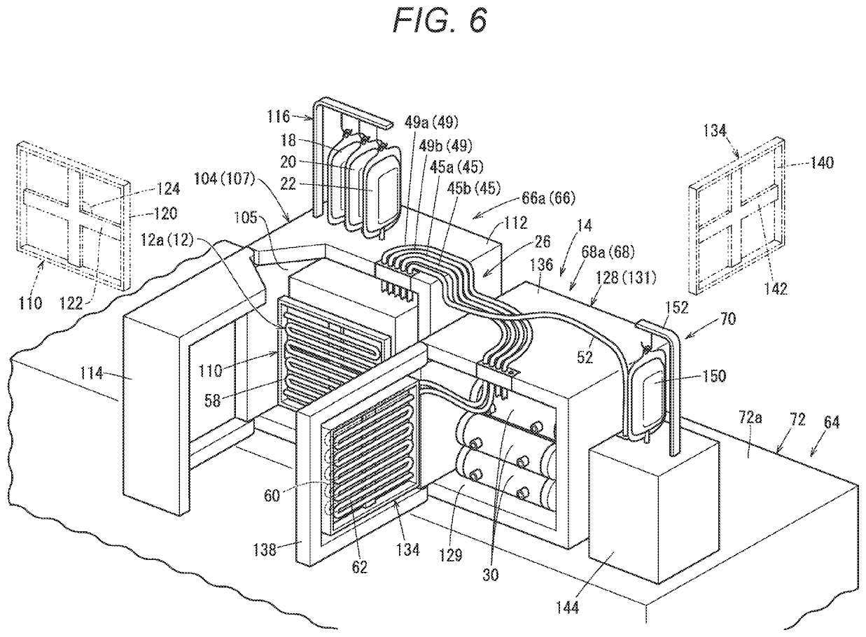

As illustrated, for example, in FIGS. 2 and 6, the first circuit control device 66a may be equipped with a box-shaped casing 104, a plurality of clamps 106, a plurality of pumps 108, and/or a first holding unit 110. As illustrated, for example, in FIG. 6, the casing 104 may include an internal space 105 in which the connection circuit 26 may be installed. The casing 104 may include a casing body 112 and a casing door 114 provided on a front surface of the casing body 112 so as to be openable and closable.

The casing 104 may have a temperature controlling function configured to keep a temperature of the internal space 105 of the casing 104 at a desired temperature (for example, about 37° C.). That is, the casing 104 may serves as a temperature raising mechanism 107 for raising temperature of the culture medium intermediate flow path 58. As illustrated, for example, in FIG. 1, a bag support unit 116 for hanging a plurality of bags (the cell solution bag 18, stripping solution bag 20, and/or collection bag 22) may be provided on an upper surface of the casing 104. On an outer surface of the casing door 114, a display unit 118 that is configured to display a current step of the cell culture and the like may be provided (refer to FIG. 1).

As illustrated, for example, in FIG. 2, the plurality of clamps 106 may include switching valves that press the wall part forming a line (tube) of the connection circuit 26 from outside to open and close an internal flow path of the line. The first circuit control device 66a may include, as the plurality of clamps 106, a first clamp 106a, a second clamp 106b, a third clamp 106c, a fourth clamp 106d, a fifth clamp 106e, a sixth clamp 106f, a seventh clamp 106g, an eighth clamp 106h, and/or a ninth clamp 106i.

The first clamp 106a may be arranged so as to be opposed to the first IC supply line 40a in the set state and may be configured to open and close an internal flow path of the first IC supply line 40a. The second clamp 106b may be arranged so as to be opposed to the second IC supply line 40b in the set state and may be configured to open and close an internal flow path of the second IC supply line 40b. The third clamp 106c may be arranged so as to be opposed to the third IC supply line 40c in the set state and may be configured to open and close an internal flow path of the third IC supply line 40c.

The fourth clamp 106d may be arranged so as to be opposed to the first EC supply line 46a in the set state and may be configured to open and close an internal flow path of the first EC supply line 46a. The fifth clamp 106e may be arranged so as to be opposed to the second EC supply line 46b in the set state and may be configured to open and close an internal flow path of the second EC supply line 46b. The sixth clamp 106f may be arranged so as to be opposed to the coupling line 50 in the set state and may be configured to open and close an internal flow path of the coupling line 50.

The seventh clamp 106g may be arranged so as to be opposed to the collection line 54 in the set state and may be configured to open and close an internal flow path of the collection line 54. The eighth clamp 106h may be arranged so as to be opposed to the IC waste liquid line 56a in the set state and may be configured to open and close an internal flow path of the IC waste liquid line 56a. The ninth clamp 106i may be arranged so as to be opposed to the EC waste liquid line 56b in the set state and may be configured to open and close an internal flow path of the EC waste liquid line 56b.

The plurality of pumps 108 may be configured to rotate so as to squeeze the wall part forming the line (tube) of the connection circuit 26, thereby imparting a flow force to the liquid inside. The circuit control device 66 may include an IC supply pump 108a and an EC supply pump 108b as the plurality of pumps 108.

The IC supply pump 108a may be arranged, in a set state, so as to be in contact with the first IC supply line 40a downstream from a coupling portion to the second IC supply line 40b and may be configured to impart a flow force in a direction toward the IC circulation circuit 44 to the liquid that flows through the first IC supply line 40a.

The EC supply pump 108b may be arranged, in a set state, so as to be in contact with the first EC supply line 46a downstream from the second EC supply line 46b and may be configured to impart a flow force in a direction toward the EC circulation circuit 48 to the liquid that flows through the second EC supply line 46b.

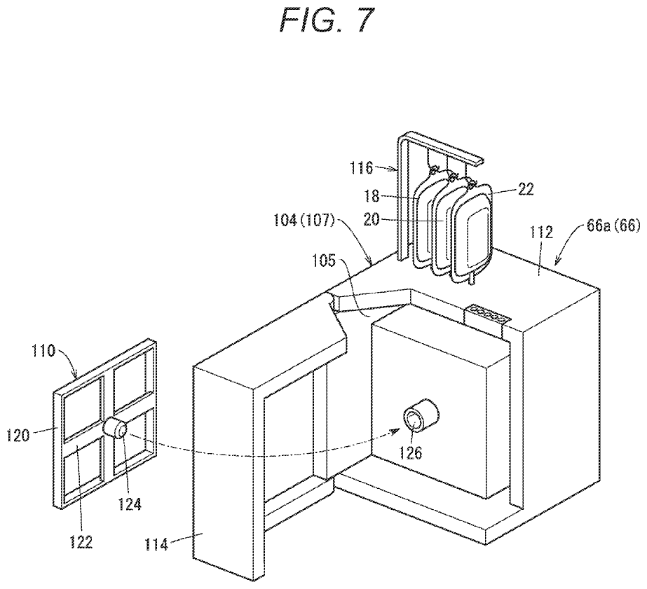

As illustrated, for example, in FIGS. 2 and 6, the first holding unit 110 may be configured to hold the culture medium intermediate flow path 58 of the culture medium supply line 42 in a predetermined shape (meandering shape). The first holding unit 110 may be provided in the internal space 105 of the casing 104. For example, as illustrated in FIGS. 6 and 7, the first holding unit 110 may include a first frame-shaped frame 120 having a quadrangular shape, a first inner frame 122 provided on the first frame-shaped frame 120, and an attachment unit 124.

The first inner frame 122 may have a cross shape. The first inner frame 122 may be coupled to a central portion of each side of the first frame-shaped frame 120. As illustrated, for example, in FIG. 6, the culture medium intermediate flow path 58 may be locked to the first frame-shaped frame 120 and the first inner frame 122 by a locking member (not illustrated) in a meandering shape. As illustrated, for example, in FIG. 7, the attachment unit 124 may be a cylindrical portion protruding from a central portion of the first inner frame 122. The attachment unit 124 may be attached to a mount unit 126 that is provided, for example, in the casing 104. The position, shape, size, and/or number of the attachment unit 124 may be appropriately changed.

As illustrated, for example, in FIG. 2, a length of the culture medium intermediate flow path 58 held by the first holding unit 110 may be set to such a length that allows the culture medium to flow for a first temperature raising time. The first temperature raising time refers to a time in which the temperature (for example, about 5° C.) of the culture medium cooled in the culture medium accommodation unit 74 is raised to desired temperature (for example, about 37° C.). The first circuit control device 66a may also include a pressure sensor, a liquid level sensor, and/or the like.

In at least one example embodiment, the mount unit 126 (refer to FIG. 7) may be formed so as to be able to rotatably support the bioreactor 30, and the first circuit control device 66a may further include an IC circulation pump 127a and an EC circulation pump 127b (refer to FIG. 2).

When the cell culturing includes using only one bioreactor (e.g., when the cell culturing is of a small amount), cell culturing may include setting the cell culturing kit that includes the only one bioreactor in the first circuit control device 66a. The bioreactor may be set in the mount unit 126.

The IC circulation pump 127a may impart a flow force in a direction toward the bioreactor to the liquid that flows through the IC circulation line of the cell culturing kit. The EC circulation pump 127b may impart a flow force in a direction toward the bioreactor to the liquid that flows through the EC circulation line of the cell culturing kit. The IC circulation pump 127a and the EC circulation pump 127b may not be needed for the cell culture that uses the cell culturing kit 12 that includes a plurality of bioreactors 30 (e.g., five bioreactors).

As illustrated, for example, in FIG. 2, the connection circuit 26 of the second cell culturing kit 12b may be set in the second circuit control device 66b. A configuration of the second circuit control device 66b may be substantially the same as the configuration of the first circuit control device 66a.

As illustrated, for example, in FIGS. 3 and 6, the processing unit 24 of the first cell culturing kit 12a may be set in the first reactor installation device 68a. The first reactor installation device 68a may be equipped with a box-shaped reactor case unit 128, five reactor support units 130, a plurality of pumps 132, and a second holding unit 134. As illustrated, for example, in FIG. 6, the reactor case unit 128 may include an internal space 129 in which the processing unit 24 (five bioreactors 30) may be installed. The reactor case unit 128 may include a reactor case body 136 and a door 138 provided on a front surface of the reactor case body 136 so as to be openable and closable. The reactor case unit 128 may have a temperature controlling function that keeps the temperature of the internal space 129 of the reactor case unit 128 at desired temperature (for example, about 37° C.). That is, the reactor case unit 128 may serve as a temperature raising mechanism 131 for raising temperature of the IC intermediate flow path 60.

As illustrated, for example, in FIG. 3, the reactor support unit 130 may be provided in the internal space 129 of the reactor case unit 128. The reactor support unit 130 may be formed so that the bioreactor 30 is attachable to and detachable from the same. The reactor support unit 130 may rotatably support the bioreactor 30 about a rotation axis Ax. The rotation axis Ax may be located at the center in an extending direction of the bioreactor 30. The rotation axis Ax may extended in a direction orthogonal to the extending direction of the bioreactor 30.

The first reactor installation device 68a may include, as plurality of pumps 132, five IC circulation pumps 132a and five EC circulation pumps 132b. The IC circulation pump 132a may be arranged so as to be in contact with the IC introduction line 44a in the set state and to impart a flow force in a direction toward the bioreactor 30 to the liquid that flows through the IC introduction line 44a. The EC circulation pump 132b may be arranged so as to be in contact with the EC introduction line 48a in the set state and to impart a flow force in a direction toward the bioreactor 30 to the liquid that flows through the EC introduction line 48a.

As illustrated, for example, in FIGS. 3 and 6, the second holding unit 134 may hold each of the IC intermediate flow path 60 of the IC introduction line 44a and the EC intermediate flow path 62 of the EC circulation line 48c in a predetermined shape (meandering shape). The second holding unit 134 may be provided in the internal space 129 of the reactor case unit 128. For example, in FIG. 6, the second holding unit 134 may include a second frame-shaped frame 140 having a quadrangular shape. A second inner frame 142 may be provided inside the second frame-shaped frame 140.

The second inner frame 142 may be formed in a cross shape. The second inner frame 142 may be coupled to a central portion of each side of the second frame-shaped frame 140. Each of the IC intermediate flow path 60 and the EC intermediate flow path 62 may be locked to the second frame-shaped frame 140 and the second inner frame 142 by a locking member (not illustrated) in a meandering shape. The second holding unit 134 may be fixed to an inner surface of the door 138.

As illustrated, for example, in FIGS. 1, 2, and 6, the first reactor installation device 68a may be provided separately from the first circuit control device 66a. For example, as illustrated, in FIGS. 2 and 6, the first cell culturing kit 12a may include an IC outer flow path 45 and an EC outer flow path 49 located outside the first circuit control device 66a and the first reactor installation device 68a in the set state. The first cell culturing kit 12a may include a first IC outer flow path 45a and a second IC outer flow path 45b as the IC outer flow path 45. As illustrated, for example, in FIG. 2, the first IC outer flow path 45a may be located on the IC circulation line 44c between a coupling portion of the first IC supply line 40a to the IC intermediate flow path 60. The second IC outer flow path 45b may be located on the IC circulation line 44c between the processing unit 24 and a coupling portion to the IC waste liquid line 56a.

The liquid that flows through the IC circulation line 44c may be cooled at positions of the first IC outer flow path 45a and the second IC outer flow path 45b. For example, the liquid that flows through the IC circulation line 44c might be cooled to about room temperature (for example, about 30° C.) at the positions of the first IC outer flow path 45a and the second IC outer flow path 45b.

A length of the IC intermediate flow path 60 held by the second holding unit 134 may be set to such a length that allows the liquid to flow for a second temperature raising time. The second temperature raising time may refer to a time in which the temperature (for example, about 30° C.) of the liquid cooled in the first IC outer flow path 45a or the second IC outer flow path 45b when flowing through the IC circulation line 44c is raised to desired temperature (the temperature of the internal space 129 of the reactor case unit 128).

The first cell culturing kit 12a may include a first EC outer flow path 49a and a second EC outer flow path 49b as the EC outer flow path 49. The first EC outer flow path 49a may be located in a section between the gas exchanger 28 and the EC intermediate flow path 62 on the EC circulation line 48c. The second EC outer flow path 49b may be located on the EC circulation line 48c between the processing unit 24 and a coupling portion to the EC waste liquid line 56b.

The liquid that flows through the EC circulation line 48c may be cooled at positions of the first EC outer flow path 49a and the second EC outer flow path 49b. For example, the liquid (culture medium) that flows through the EC circulation line 48c might be cooled to about room temperature (for example, about 30° C.) at the positions of the first EC outer flow path 49a and the second EC outer flow path 49b.

A length of the EC intermediate flow path 62 held by the second holding unit 134 may be set to such a length that allows the liquid to flow for a third temperature raising time. The third temperature raising time may refer to a time in which the temperature (for example, about 30° C.) of the liquid cooled in the first EC outer flow path 49a or the second EC outer flow path 49b when flowing through the EC circulation line 48c is raised to desired temperature (the temperature of the internal space 129 of the reactor case unit 128).

The processing unit 24 of the second cell culturing kit 12b may be installed (set) in the second reactor installation device 68b. A configuration of the second reactor installation device 68b may be substantially the same as the configuration of the first reactor installation device 68a.

As illustrated, for example, in FIG. 2, the sensor device 70 may be connected to the first cell culturing kit 12a and the second cell culturing kit 12b in the set state. The sensor device 70 may include a box-shaped sensor case unit 144 (refer to FIGS. 1 and 6), two pumps 146, a sensor unit 148, and a waste liquid bag 150. A bag support unit 152 for hanging the waste liquid bag 150 may be provided on an upper surface of the sensor case unit 144 (refer to FIGS. 1 and 6). The two pumps 146 and the sensor unit 148 may be arranged in the sensor case unit 144.

The pump 146 may be formed similarly to the pump 108 described above. The sensor device 70 may include a first sampling pump 146a and a second sampling pump 146b as the two pumps 146. The first sampling pump 146a may be arranged so as to be in contact with the sampling line 52 of the first cell culturing kit 12a in the set state and to impart a flow force in a direction toward the sensor unit 148 to the liquid (culture medium) that flows through the sampling line 52. The second sampling pump 146b may be arranged so as to be in contact with the sampling line 52 of the second cell culturing kit 12b in the set state and to impart a flow force in a direction toward the sensor unit 148 to the liquid (culture medium) that flows through the sampling line 52.

The sensor unit 148 may be configured to measure components (concentration of pH, O₂, CO₂, glucose, lactic acid, and/or the like) of the culture medium as guided by the sampling line 52. The culture medium after the measurement by the sensor unit 148 is completed may be discharged to the waste liquid bag 150.

In the cell culturing device 14, the sensor device 70 (the sensor unit 148 and the waste liquid bag 150) may be shared by the first cell culturing kit 12a and the second cell culturing kit 12b. The tank device 64 may be shared by the first cell culturing kit 12a and the second cell culturing kit 12b.

As illustrated, for example, in FIG. 1, the controller 16 may include a computer that includes a processor, a memory, and an input/output interface. The controller 16 may perform overall control of an entire system by the processor executing a program stored in the memory. The controller 16 may be connected to the first circuit control device 66a, the first reactor installation device 68a, the second circuit control device 66b, the second reactor installation device 68b, and the sensor device 70 by wire, wirelessly, via a network, or a communication means obtained by combing them.

The first circuit control device 66a and the second circuit control device 66b may both control operations of the plurality of clamps 106 and the plurality of pumps 108 on the basis of a control signal(s) from the controller 16. The first reactor installation device 68a and the second reactor installation device 68b may both control operations of the plurality of IC circulation pumps 132a and the plurality of EC circulation pumps 132b, and also a rotation operation of each bioreactor 30, on the basis a control signal(s) from the controller 16.

The sensor unit 148 may obtain (sample) the culture medium that flows through the first cell culturing kit 12a or the second cell culturing kit 12b on the basis of a control signal(s) from the controller 16. The sensor unit 148 may be configured to measure the components of the obtained culture medium. The sensor unit 148 may transmit a measurement result to the controller 16. The controller 16 may estimate the number of cells cultured in the first cell culturing kit 12a and the second cell culturing kit 12b on the basis of the measurement result. The controller 16 feedback-controls each operation of the first circuit control device 66a, the first reactor installation device 68a, the second circuit control device 66b, and the second reactor installation device 68b on the basis of the measurement result from the sensor device 70.

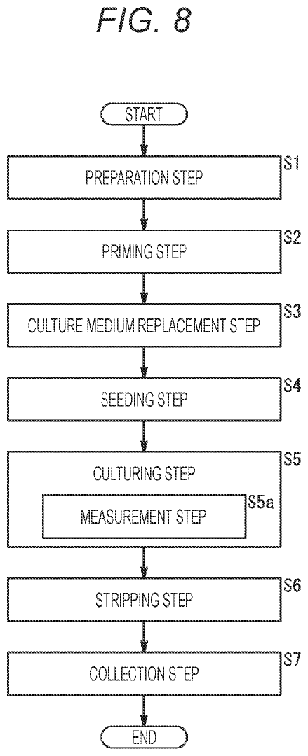

As illustrated, for example, in FIG. 8, a cell culturing method using, for example, the cell culturing system 10, may include a preparation step, a priming step, a culture medium replacement step, a seeding step, a culturing step, a stripping step, and/or a collection step.

With reference to FIGS. 2 and 8, during the preparation step (step S1), the culture medium accommodation unit 74 may be arranged in the first case unit 77 and the cleaning solution accommodation unit 76 and the waste liquid accommodation unit 78 may be arranged in the second case unit 80. The processing unit 24 (five bioreactors 30) of the first cell culturing kit 12a may be installed in the first reactor installation device 68a and the connection circuit 26 of the first cell culturing kit 12a may be set in the first circuit control device 66a. The plurality of bags (e.g., the cell solution bag 18, stripping solution bag 20, and/or collection bag 22) of the first cell culturing kit 12a may be hung on the bag support unit 116 of the first circuit control device 66a. The connection circuit 26 of the first cell culturing kit 12a may be aseptically joined to each of the culture medium accommodation unit 74, the cleaning solution accommodation unit 76, the waste liquid accommodation unit 78, and the sensor unit 148.

Subsequently, the processing unit 24 (five bioreactors 30) of the second cell culturing kit 12b may be installed in the second reactor installation device 68b, and the connection circuit 26 of the second cell culturing kit 12b may be set in the second circuit control device 66b. At that time, the plurality of bags (e.g., the cell solution bag 18, stripping solution bag 20, and/or collection bag 22) of the second cell culturing kit 12b may be hung on the bag support unit 116 of the second circuit control device 66b. The connection circuit 26 of the second cell culturing kit 12b may be aseptically joined to each of the culture medium accommodation unit 74, the cleaning solution accommodation unit 76, the waste liquid accommodation unit 78, and the sensor unit 148.

During the priming step (step S2), the circuit control device 66 and the reactor installation device 68 may be configured to drive predetermined clamp 106 and pumps 108 and 132 to guide the cleaning solution from the cleaning solution accommodation unit 76 to the connection circuit 26 and each bioreactor 30. The connection circuit 26 and each bioreactor 30 (IC region and EC region) may be filled with the cleaning solution and the air present in the connection circuit 26 and the bioreactor 30 may be discharged to the waste liquid accommodation unit 78 together with the cleaning solution.

During the culture medium replacement step (step S3), the circuit control device 66 and the reactor installation device 68 may be configured to drive the predetermined clamp 106 and pumps 108 and 132 to guide the culture medium in the culture medium accommodation unit 74 to the connection circuit 26 and each bioreactor 30. As a result, the cleaning solution present in the connection circuit 26 and each bioreactor 30 (IC region and EC region) may be replaced with the culture medium.

During the seeding step (step S4), the circuit control device 66 and the reactor installation device 68 may be configured to drive the predetermined clamp 106 and pumps 108 and 132 to supply the cell solution in the cell solution bag 18 to the IC region of each bioreactor 30. For example, the cell solution guided from the cell solution bag 18 to the IC circulation line 44c via the first IC supply line 40a may be divided into the five IC introduction lines 44a and guided to the IC region of each bioreactor 30 (refer to FIG. 3). At that time, since the five IC circulation pumps 132a impart a flow force to the liquid (cell solution) that flows through the five IC introduction lines 44a, the cell solution may be supplied substantially uniformly to the five bioreactors 30.

During the culturing step (step S5), the circuit control device 66 and the reactor installation device 68 may be configured to drive the predetermined clamp 106 and pumps 108 and 132 to supply the culture medium in the culture medium accommodation unit 74 to the IC region and the EC region of each bioreactor 30 and culture (propagate) the cells in the hollow fiber 32 of the bioreactor 30. It should be appreciated that the supply of the culture medium to the IC region of each bioreactor 30 and the supply of the culture medium to the EC region of each bioreactor 30 may be performed simultaneously or separately. During the culturing step, it may be possible that the culture medium is not supplied to the IC region of each bioreactor 30 and that the culture medium is supplied only to the EC region of each bioreactor 30.

During the culturing step, a low-temperature (for example, about 5° C.) culture medium from the culture medium accommodation unit 74 may flow through the culture medium supply line 42 and may be guided from the tank device 64 to the culture medium intermediate flow path 58 provided in the internal space 105 of the casing 104 of the circuit control device 66. The temperature of the culture medium that flows through the culture medium intermediate flow path 58 may be raised to a desired temperature (for example, about 37° C.)

In a case where the culture medium is supplied to the IC region of each bioreactor 30, the culture medium of which temperature may be raised in the culture medium intermediate flow path 58 and introduced into the IC circulation line 44c via the third IC supply line 40c, the second IC supply line 40b, and the first IC supply line 40a. The temperature of the culture medium introduced into the IC circulation line 44c may be lowered when the culture medium flows through the first IC outer flow path 45a (for example, lowered to about 30° C.)

Thereafter, the culture medium the temperature of which is lowered may be guided to the IC intermediate flow path 60 provided in the internal space 129 of the reactor case unit 128. The temperature of the culture medium that flows through the IC intermediate flow path 60 may be raised to desired temperature (for example, about 37° C.). The culture medium that flows through the IC intermediate flow path 60 may be branched into the five IC introduction lines 44a and guided to the IC region of each bioreactor 30 so that the culture medium of the IC region of each bioreactor 30 may be replaced with a new one. As a result, nutrients (such as oxygen) may be efficiently supplied to the cells seeded on an inner surface of the hollow fiber 32 in each bioreactor 30.

During the culturing step, the culture medium may circulate in the IC circulation circuit 44. At that time, the temperature of the culture medium may be lowered when the culture medium flows through the first IC outer flow path 45a and the second IC outer flow path 45b but may be raised in the IC intermediate flow path 60 so that the temperature of the culture medium supplied to the IC region of each bioreactor 30 may be kept at desired temperature.

When the culture medium is supplied to the EC region of each bioreactor 30, the culture medium of which temperature is raised in the culture medium intermediate flow path 58 may be introduced into the EC circulation line 48c via the first EC supply line 46a. The temperature of the culture medium introduced into the EC circulation line 48c may be lowered when the culture medium flows through the first EC outer flow path 49a after passing through the gas exchanger 28 (for example, lowered to about 30° C.).

Thereafter, the culture medium the temperature of which is lowered may be guided to the EC intermediate flow path 62 provided in the internal space 129 of the reactor case unit 128. The temperature of the culture medium that flows through the EC intermediate flow path 62 may be raised to a desired temperature (for example, about 37° C.). The culture medium that flows through the EC intermediate flow path 62 may branched into the five EC introduction lines 48a and may be guided to the EC region of each bioreactor 30. In each bioreactor 30, nutrients and the like may be exchanged between the culture medium of the IC region and the culture medium of the EC region. As a result, nutrients (such as oxygen) may be efficiently supplied to the cells seeded on an inner surface of the hollow fiber 32 in each bioreactor 30.

During the culturing step, the culture medium circulates in the EC circulation circuit 48. The temperature of the culture medium may be lowered when the culture medium flows through the first EC outer flow path 49a and the second EC outer flow path 49b but may be raised in the EC intermediate flow path 62 so that the temperature of the culture medium supplied to the EC region of each bioreactor 30 is kept at desired temperature. The culture medium that circulates in the EC circulation circuit 48 may be subjected to gas exchange when flowing through the gas exchanger 28. Therefore, the culture medium containing a desired gas component may be supplied to the EC region of each bioreactor 30.

The culturing step may further include a measurement step (step S5a). During the measurement step, the sensor device 70 may drive the pump 146 to guide the culture medium that flows through the EC circulation line 48c downstream of the processing unit 24 to the sensor unit 148. The sensor unit 148 may be configured to measure components of the culture medium (culture medium in the processing unit 24). The measurement result of the sensor unit 148 may be transmitted to the controller 16. The controller 16 may determines a time (timing), a period, the number of times, and/or the like of replacement of the culture medium on the basis of the measurement result. The culture medium after the measurement by the sensor unit 148 is completed may be discharged to the waste liquid bag 150. The time (timing), the number of times, and/or the like of the measurement step performed during the culturing step may be appropriately set.

When the culturing step is finished, during the stripping step (step S6), the circuit control device 66 and the reactor installation device 68 may drive the predetermined clamp 106 and pumps 108 and 132 to guide the stripping solution to the IC region of each bioreactor 30. As a result, the cells cultured (propagated) in the IC region of each bioreactor 30 may be stripped from the inner surface of the hollow fiber 32.

Subsequently, during the collection step (step S7), the circuit control device 66 and the reactor installation device 68 may drive the predetermined clamp 106 and pumps 108 and 132 to guide the cells stripped at the stripping step from each bioreactor 30 to the collection bag 22 while supplying the culture medium to the IC region of each bioreactor 30. After the collection step is completed, the operation of the current cell culturing method may be finished.

The cell culturing system 10 may include the processing unit 24 that cultures the cells, the reactor installation device 68 in which the processing unit 24 may be installed, the connection circuit 26 connected to the processing unit 24, a plurality of circuit control devices 66 to and from which the connection circuit 26 is attachable and detachable for supplying the cells and culture medium from the connection circuit 26 to the processing unit 24 and collecting the cultured cells from the processing unit 24 to the connection circuit 26, and the tank device 64 including the culture medium accommodation unit 74 for accommodating the culture medium. The processing unit 24 may include a plurality of bioreactors 30. The culture medium of the culture medium accommodation unit 74 may be supplied to the processing unit 24 via the connection circuit 26. The culture medium accommodation unit 74 may accommodate the culture medium of the amount required for culturing the cells in the processing units 24.

According to such a configuration, it may be sufficient to prepare the circuit control device 66 for each processing unit 24 (the plurality of bioreactors 30), so that the number of the circuit control devices 66 may be smaller than the number of the bioreactors 30, efficiently increasing the amount of cell culture while suppressing an increase in cost. Since the culture medium accommodation unit 74 may accommodate the culture medium of the amount required for culturing the cells in the processing unit 24, even when a large amount of culture medium is required at the time of cell culture using the plurality of bioreactors 30, it may not be required to replace the culture medium accommodation unit 74 during the cell culture, such that the cell culture may be performed smoothly and efficiently.

The tank device 64 may be configured to cool the culture medium in the culture medium accommodation unit 74.

It may be possible to suppress denaturation of the components of the culture medium in the culture medium accommodation unit 74 during the cell culturing period.

The connection circuit 26 may include the culture medium intermediate flow path 58 provided between the culture medium accommodation unit 74 and the processing unit 24. The cell culturing system 10 may include a temperature raising mechanism 107 for raising the temperature of the culture medium intermediate flow path 58.

According to such a configuration, it may be possible to raise the temperature of the low-temperature culture medium guided from the culture medium accommodation unit 74 when this flows through the culture medium intermediate flow path 58. Therefore, temperature lowering of the culture medium in each bioreactor 30 (IC region and EC region) may be suppressed.

The circuit control device 66 may include the casing 104 that may include the internal space 105 kept at desired temperature and that serves as the temperature raising mechanism 107. The temperature of the culture medium intermediate flow path 58 may be raised when arranged in the internal space 105 of the casing 104.

According to such a configuration, it may not be required to prepare a temperature raising device for raising the temperature of the culture medium intermediate flow path 58 separately from the casing 104, reducing the cost and limiting the complexity of the circuit control device 66.

A flow path length of the culture medium intermediate flow path 58 may be set to such a length that the temperature of the culture medium guided from the culture medium accommodation unit 74 may be raised to the temperature of the internal space 105 of the casing 104 when this flows through the culture medium intermediate flow path 58.

According to such a configuration, it may be possible to raise the temperature of the culture medium that flows through the culture medium intermediate flow path 58 to the temperature of the internal space 105 of the casing 104.

The culture medium intermediate flow path 58 may extended linearly. The cell culturing system 10 may include the first holding unit 110 that holds the culture medium intermediate flow path 58 in a meandering state.

According to such a configuration, it may be possible to compactly arrange the culture medium intermediate flow path 58 in the internal space 105 of the casing 104. It may be possible to suppress the culture medium intermediate flow path 58 from being bent to close the flow path.

The culture medium accommodation unit 74 may include the culture medium tank 90 that accommodates the culture medium and the culture medium installing member 92 in which the culture medium tank 90 is installed. The roller 94 for moving the culture medium installing member 92 may be provided on the bottom surface of the culture medium installing member 92.

According to such a configuration, the culture medium accommodation unit 74 may be easily taken in and out of the tank device 64.

The cell culturing system 10 may be equipped with the sensor device 70 for measuring the components of the culture medium guided to the processing unit 24.

According to such a configuration, since the components of the culture medium of the processing unit 24 may be measured by the sensor device 70, the cell culture may be efficiently performed.

The cell culturing system 10 may include the controller 16 that controls the operation of the circuit control device 66. The controller 16 feedback-controls the operation of the circuit control device 66 on the basis of the measurement result of the sensor device 70.

Each of a plurality of bioreactors 30 may include a plurality of hollow fibers 32.

The cell culturing system 10 is not limited to the above-described configuration. For example, the number of the bioreactors 30 that may be installed by the reactor installation device 68 is not limited to five and in other embodiments may include, for example, two, three, four, six, or more. In the cell culturing system 10, three or more circuit control devices 66 and three or more reactor installation devices 68 may be provided. In this case, two or more tank devices 64 and two or more sensor devices 70 may be provided.

In the cell culturing system 10, the IC intermediate flow path 60 or the EC intermediate flow path 62 may be omitted. In the cell culturing system 10, both the IC intermediate flow path 60 and the EC intermediate flow path 62 may be omitted, and the second holding unit 134 may be omitted. Furthermore, in the cell culturing system 10, the culture medium intermediate flow path 58 and the first holding unit 110 may be omitted.

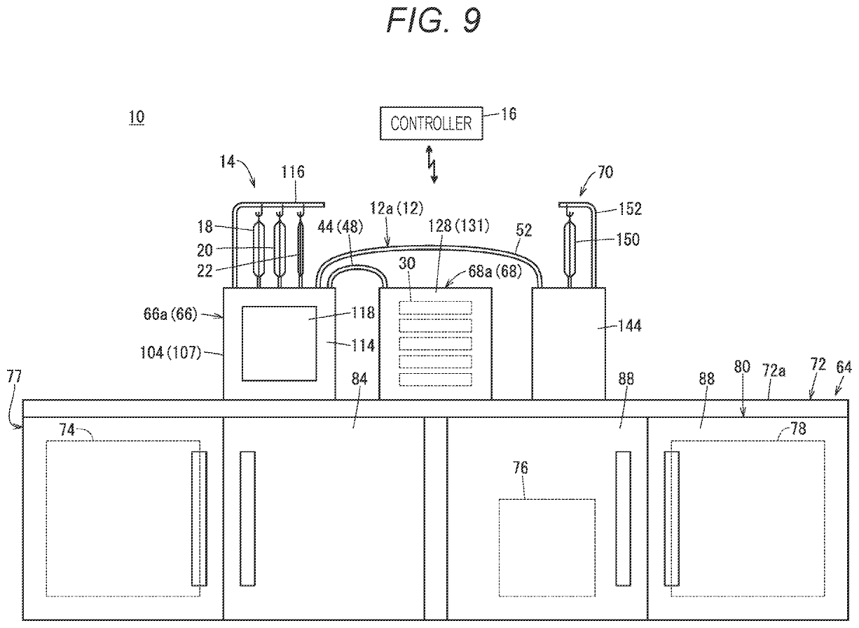

As illustrated, for example, in FIG. 9, it the cell culturing device 14 may be equipped with the tank device 64, the first circuit control device 66a (one circuit control device 66), the first reactor installation device 68a (one reactor installation device 68), and the sensor device 70, and the second circuit control device 66b and the second reactor installation device 68b may be omitted.

A cell culturing system 10A according to another example embodiment may include some of the same components as those of the cell culturing system 10 described above and are denoted by the same reference numerals.

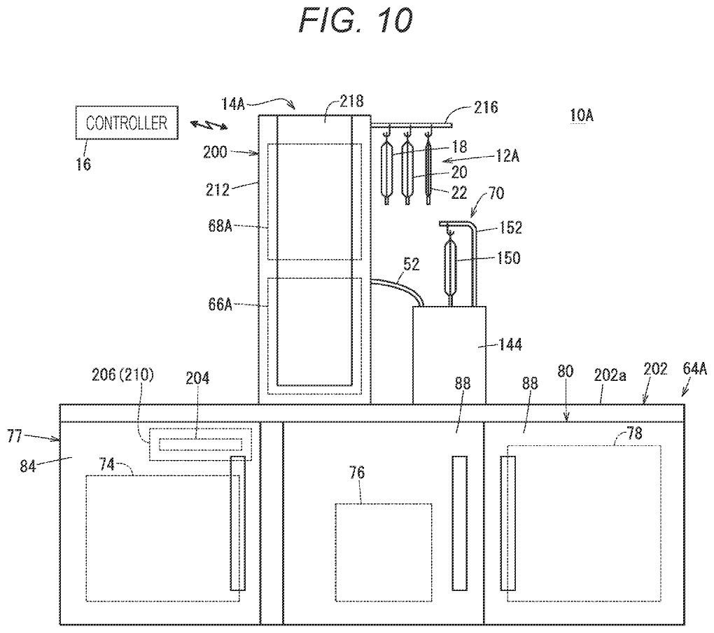

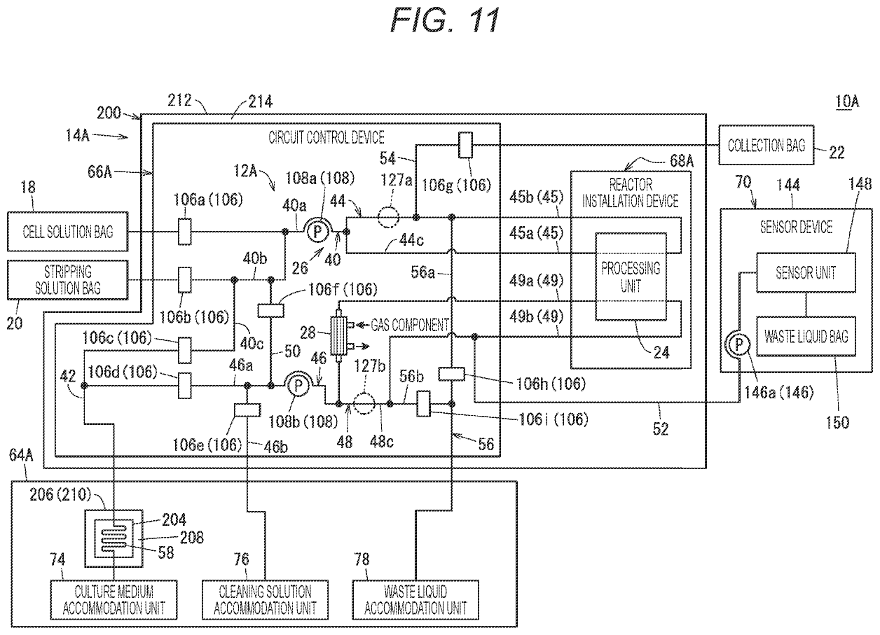

As illustrated, for example, in FIGS. 10 and 11, the cell culturing system 10A may include one cell culturing kit 12A, a cell culturing device 14A, and the controller 16. The cell culturing kit 12A may be formed similarly to the cell culturing kit 12 described above except that the IC intermediate flow path 60 and the EC intermediate flow path 62 are not included.

The cell culturing device 14A may include one tank device 64A, a culturing device body 200, and one sensor device 70. The tank device 64A may include a base 202, the culture medium accommodation unit 74, the cleaning solution accommodation unit 76, the waste liquid accommodation unit 78, and a holding unit 204. The culturing device body 200 and the sensor device 70 may be arranged so as to be adjacent to each other on an upper surface 202a of the base 202. The base 202 may include the first case unit 77, the second case unit 80, and a third case unit 206.

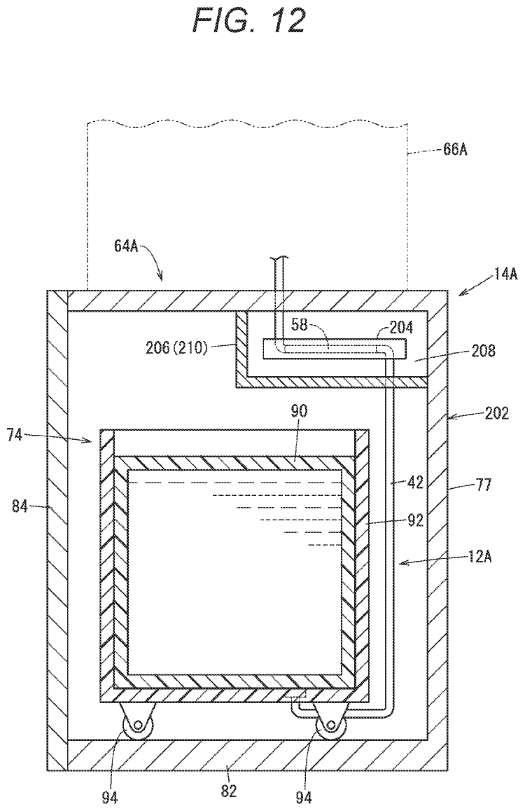

As illustrated, for example, in FIG. 12, the third case unit 206 may be provided on an upper portion of the first case unit 77. A position of the third case unit 206 may be appropriately set. The third case unit 206 may have a temperature controlling function of keeping temperature of an internal space 208 of the third case unit 206 at desired temperature (for example, about 37° C.). The holding unit 204 may be configured to hold the culture medium intermediate flow path 58 in a predetermined shape (meandering shape). The holding unit 204 may be provided in the internal space 208 of the third case unit 206. That is, the third case unit 206 may serve as a temperature raising mechanism 210 for raising the temperature of the culture medium intermediate flow path 58. The holding unit 204 may be formed similarly to the first holding unit 110 and/or the second holding unit 134 as described above, for example.

As illustrated, for example, in FIGS. 10 and 11, the culturing device body 200 may include a box-shaped case member 212, a circuit control device 66A, and a reactor installation device 68A arranged in an internal space 214 of the case member 212. The culturing device body 200 may also include a bag support unit 216 provided on the case member 212. That is, the circuit control device 66A and the reactor installation device 68A may not be provided separately, but rather integrally provided in the internal space 214 of the case member 212. A display unit 218 may be provided on a front surface of the case member 212.

As illustrated, for example, in FIG. 11, the circuit control device 66A may be equipped with a plurality of clamps 106 and a plurality of pumps 108, similar to the circuit control device 66 described above. The circuit control device 66A may not include the casing 104 and the first holding unit 110 described above. In the reactor installation device 68A, the processing unit 24 (five bioreactors 30) may be installed. As is the case with the reactor installation device 68 described above, the reactor installation device 68A may be equipped with the five reactor support units 130 and a plurality of pumps 132 (refer to FIG. 3). The reactor installation device 68A may not include the reactor case unit 128 and the second holding unit 134 described above (refer to FIG. 2). The bag support unit 216 may be formed in such a manner that a plurality of bags (e.g., cell solution bag 18, stripping solution bag 20, and/or collection bag 22) may be hung.

In the cell culturing system 10A, the temperature raising mechanism 210 may be provided in the tank device 64A. According to such a configuration, the configuration of the circuit control device 66A may be made compact as compared with, for example, an instance where the temperature raising mechanism 210 is provided in the circuit control device 66A.

It should be appreciated that the present disclosure is not limited to the above-described embodiments and various modifications may be made in accordance with different aspects of the disclosure.

In at least one example embodiment, a cell culturing system (10, 10A) may include a processing unit (24) that cultures cells, a reactor installation device (68, 68A) in which the processing unit may be installable, a connection circuit (26) connected to the processing unit, a circuit control device (66, 66A) to and from which the connection circuit may be attachable and detachable, the circuit control device for supplying the cells and a culture medium from the connection circuit to the processing unit and collecting cultured cells from the processing unit to the connection circuit, and a tank device (64, 64A) including a culture medium accommodation unit (74) for accommodating the culture medium. The processing unit may include a plurality of bioreactors (30), the culture medium in the culture medium accommodation unit may be supplied to the processing unit via the connection circuit, and the culture medium accommodation unit may be capable of accommodating the culture medium of an amount required for culturing the cells in the processing unit.

The tank device may be configured to cool the culture medium in the culture medium accommodation unit.

The connection circuit may include a culture medium intermediate flow path (58) provided between the culture medium accommodation unit and the processing unit. The cell culturing system may include a temperature raising mechanism (107, 210) configured to raise temperature of the culture medium intermediate flow path.

The temperature raising mechanism may be provided in the tank device or the circuit control device.

The circuit control device may include a casing (104) including an internal space (105) kept at desired temperature and serving as the temperature raising mechanism. The temperature of the culture medium intermediate flow path may be raised when the culture medium intermediate flow path is arranged in the internal space of the casing.

A flow path length of the culture medium intermediate flow path may be set to such a length that a temperature of the culture medium guided from the culture medium accommodation unit may be raised to the temperature of the internal space of the casing when the culture medium flows through the culture medium intermediate flow path.

The culture medium intermediate flow path may be extended linearly. The cell culturing system may include a holding unit (110, 204) that is configured to hold the culture medium intermediate flow path in a meandering state.

The culture medium accommodation unit may include a culture medium tank (90) in which the culture medium may be accommodated, a culture medium installing member (92) in which the culture medium tank may be installed, and a roller (94) for moving the culture medium installing member on a bottom surface of the culture medium installing member.

The cell culturing system may also include a sensor device (70) configured to measure a component of the culture medium guided to the processing unit.

The cell culturing system may also include a controller (16) that is configured to control an operation of the circuit control device. The controller may feedback-control the operation of the circuit control device on the basis of a measurement result of the sensor device.

Each of the plurality of bioreactors may include a plurality of hollow fibers (32).

1. A cell culturing system comprising:
one or more processing units, each processing unit including a plurality of bioreactors; and
one or more circuit control devices, each circuit control device configured to supply the cells and a culture medium to at least one of the one or more processing units and to collect cultured cells from at least one of the one or more processing units, a number of bioreactors being greater than a number of circuit control devices.

2. The cell culturing system of claim 1, wherein the cell culturing system further includes:
a connection circuit connected to the one or more processing units and removably coupled to the one or more circuit control devices.

3. The cell culturing system of claim 2, wherein the cell culturing system further includes:
a tank device including a culture medium accommodation unit that is configured to hold the culture medium, the connection circuit connecting the culture medium accommodation unit and the one or more circuit control devices.

4. The cell culturing system of claim 3, wherein the culture medium accommodation unit is configured to accommodate an amount of the culture medium required for culturing the cells in each of the one or more processing units.

5. The cell culturing system of claim 3, wherein the culture medium accommodation unit includes:
a culture medium tank in which the culture medium is accommodated; and
a culture medium installing member in which the culture medium tank is installed.

6. The cell culturing system of claim 5, wherein the culture medium accommodation unit further includes a roller disposed on a bottom surface of the culture medium installing member.

7. The cell culturing system of claim 3, wherein the tank device is configured to cool the culture medium in the culture medium accommodation unit.

8. The cell culturing system of claim 3, wherein the connection circuit includes a culture medium intermediate flow path that connects the culture medium accommodation unit and at least one of the one or more processing units.

9. The cell culturing system of claim 8, wherein the cell culturing system further includes:
a holding unit that holds the culture medium intermediate flow path in a meandering state.

10. The cell culturing system of claim 8, wherein the cell culturing system further includes:
a temperature raising mechanism for raising a temperature of the culture medium intermediate flow path.

11. The cell culturing system of claim 10, wherein the temperature raising mechanism is provided in the tank device or the circuit control device.

12. The cell culturing system of claim 10, wherein each of the one or more circuit control devices includes a casing defining an internal space that is maintained at a selected temperature, the casing serving as the temperature raising mechanism, a temperature of the culture medium intermediate flow path raised when the culture medium intermediate flow path is arranged in the internal space of the casing.

13. The cell culturing system of claim 12, wherein the culture medium intermediate flow path has a flow path length such that the temperature of the culture medium as guided from the culture medium accommodation unit is raised to the selected temperature of the internal space of the casing when the culture medium flows through the culture medium intermediate flow path.

14. The cell culturing system of claim 1, wherein the cell culturing system further includes:
one or more reactor installation devices configured to receive at least one of the one or more processing units.

15. The cell culturing system of claim 1, wherein the cell culturing system further includes:
a controller that is configured to control operations of at least one of the one or more circuit control devices.

16. The cell culturing system of claim 15, wherein the cell culturing system further includes:
a sensor device for measuring a component of the culture medium as guided to at least one of the one or more processing units.

17. The cell culturing system of claim 16, wherein
the controller feedback-controls the operation of the at least one of the one or more circuit control devices on the basis of a measurement result of the sensor device.

18. The cell culturing system of claim 1, wherein each of the plurality of bioreactors includes a plurality of hollow fibers.

19. A cell culturing system comprising:
a processing unit including a plurality of bioreactors;
a circuit control device configured to supply the cells and a culture medium to the processing unit and to collect cultured cells from the processing unit;
a connection circuit connected to the processing unit and removably coupled to the circuit control device;
a tank device including a culture medium accommodation unit and is configured to hold the culture medium in a cool state, the connection circuit including a culture medium intermediate flow path connecting the culture medium accommodation unit and the circuit control device, the culture medium accommodation unit being configured to accommodate an amount of the culture medium required for culturing the cells in the processing unit; and
a temperature raising mechanism for raising a temperature of the culture medium intermediate flow path.

20. The cell culturing system of claim 19, wherein the circuit control device includes a casing defining an internal space that is maintained at a selected temperature, the casing serving as the temperature raising mechanism, a temperature of the culture medium intermediate flow path being raised when the culture medium intermediate flow path is arranged in the internal space of the casing, and the culture medium intermediate flow path having a flow path length such that the temperature of the culture medium as guided from the culture medium accommodation unit is raised to the selected temperature of the internal space of the casing when the culture medium flows through the culture medium intermediate flow path.
